(19) United States
(12) Patent Application Publication
(10) Pub. No.: US 2010/0143257 A1
(43) Pub. Date: Jun. 10, 2010

(54) Prevention of Cancers by Immunization

(76) Inventors: Boris Skurkovich, Pawtucket, RI (US); Ellen Millstein, San Diego, CA (US); Simon Skurkovich, Rockville, MD (US); Melvin Millstein, San Diego, CA (US)

Correspondence Address: HOUSTON ELISEEVA, 4 MILITIA DRIVE, SUITE 4, LEXINGTON, MA, 02421, US

(21) Appl. No.: 12/612,030

(22) Filed: Nov. 4, 2009

Related U.S. Application Data
(63) Continuation of application No. PCT/US2008/062452, filed on May 2, 2008.
(60) Provisional application No. 60/915,963, filed on May 4, 2007.

(51) Int. Cl. A61K 39/00; A61K 49/00; A61P 35/00

(52) U.S. Cl. 424/9.2; 424/277.1

(57) ABSTRACT

In healthy subjects, especially the ones with increased risk for developing cancer due to genetic predisposition or other risk factors, cancer can be prevented by immunization with a cancer vaccine. Oncoantigens present in the vaccine preferably are incubated with IFN-γ, IL-2 or both. To prevent cancer recurrence, cancer patients in remission are given a prolonged course of immunizations with a vaccine that includes, for example, oncoantigens in combination with immune competent cells (e.g., bone marrow and T lymphocytes). The bone marrow and T lymphocytes are optionally treated with IFN-γ, IL-2 or both.

### RELATED APPLICATIONS

This application is a Continuation of PCT Application Number PCT/US2008/062452 filed on May 2, 2008, which claims the benefit under 35 USC 119(e) of U.S. Provisional Application No. 60/915,963, filed on May 4, 2007, both of which are incorporated herein by reference in their entirety.

### BACKGROUND OF THE INVENTION

Cancer remains a very serious medical condition and in many cases the disease progresses to a fatal outcome.

In some cases, the patients have had solid tumor mass removed by surgery. As known in the art, surgical techniques often fail to remove all traces of cancer and cancer cells left can lead to reemergence of cancer. While chemotherapeutic and radiation techniques often lead to remission, they often do not offer a permanent solution and have serious side effects. Results with non-conventional approaches, e.g., herbal remedies remain controversial and hormone or combination therapies also are accompanied by disadvantages. In spite of advances made in surgical techniques, radiation, chemotherapy and other approaches, a need continues to exits for improved methods for treating cancer.

Based on factors such as family history, genetic predisposition, occupation, and exposure to certain agents some people are statistically more prone to develop cancer than others. Accordingly, a need also exists for methods designed to prevent the disease.

### SUMMARY OF THE INVENTION

The invention generally relates to building up the immune system and immune response in cancer prevention in healthy subjects or to prevent recurrence of an existing cancer. It is particularly useful in preventing onset and development of malignant solid tumors, e.g., cancer of the breast, ovary, prostate and so forth. Preferred examples of the invention are carried out utilizing vaccines that include, consist essentially of, or consist of (i) a prototypic cancer antigen; (ii) allogeneic whole cancer cells or (iii) autologous whole cancer cells. Suitable vaccines also can be obtained from one or more sources such as lysed tumor cells, gene-modified tumor cells, heat-shock proteins, peptides, naked DNA, ex vivo dendritic cells, and others. Typically present in the vaccines are oncoantigens such as cells, cell extracts, DNA, polypeptides and so forth.

In one aspect, a method for cancer prevention comprises selecting a healthy subject who is cancer-free, has never been diagnosed with cancer, and has an intact immune system; and administering to the subject a vaccine. In some embodiments, the vaccine includes a prototypic cancer antigen, preferably one that is naturally expressed in a wide variety of cancer types. In other embodiments, the vaccine includes, or is obtained from, cancer cells collected from a matched donor. In preferred embodiments, the cancer vaccine is treated with IFN-γ, IL-2 or both prior to administration. In specific implementations, the vaccine is administered to subjects who are at risk for developing cancer.

In another aspect, a method for preventing cancer recurrence comprises selecting a cancer patient who is in remission for at least ten months; and administering to the patient a vaccine that includes a prototypic cancer antigen, e.g., NY-ESO-1, that is naturally expressed in a wide variety of cancer types, in combination with bone marrow, and, optionally, T lymphocytes, thereby preventing cancer recurrence. One or more of the cancer antigen, the bone marrow and optional T lymphocytes can be treated with IFN-γ, IL-2 or both. In preferred implementations, the bone marrow is pretreated with a combination of IFN-γ and IL-2. In another preferred implementation, T lymphocytes are pretreated with IFN-γ. In yet another preferred implementation, the cancer antigen is pretreated with IFN-γ, IL-2 or both.

In a further aspect, a method for preventing cancer recurrence comprises selecting a cancer patient who is in remission for at least ten months; and administering to the patient a vaccine that includes, or is obtained from, autologous cancer cells, in combination with bone marrow, and, optionally, T lymphocytes, thereby preventing cancer recurrence. One or more of: the cancer cells, their extracts, the bone marrow and/or optional T lymphocytes can be pretreated with IFN-γ, IL-2 or both. In preferred implementations, the bone marrow is pretreated with a combination of IFN-γ and IL-2. In another preferred implementation, T lymphocytes are pretreated with IFN-γ. Yet in another preferred implementation, cancer antigen present in the cancer cells or extracts thereof is treated with IFN-γ, IL-2 or both.

In yet another aspect, a method for conducting an experiment in animals or in a clinical trial comprises selecting a group of healthy subjects who have intact immune system and who are at risk for developing a specific type of cancer in the future; administering to the healthy subjects a vaccine consisting of a prototypic cancer antigen to obtain an immunized group; monitoring the immunized group with respect to immune indicators or with respect to onset of said type of cancer; and comparing the immunized group with a group that has not received the vaccine with respect to incidence of said immune indicators or the incidence of the onset of said type of cancer.

In still another aspect, a method for conducting an experiment in animals or in a clinical trial comprises selecting a group of healthy subjects who have intact immune system and who are at risk for developing a specific type of cancer in the future; administering to the healthy subjects a vaccine obtained from cells collected from one or more matched donor(s) to obtain an immunized group; monitoring the immunized group with respect to immune indicators or with respect to onset of said type of cancer; and comparing the immunized group with a group that has not received the vaccine with respect to incidence of said immune indicators or the incidence of the onset of said type of cancer.

In yet other aspects, the invention is directed to use of allogeneic cells obtained from one or more matched donors for preventing cancer in a healthy subject; use of autologous cells obtained from a cancer patient in combination with bone marrow and/or collected from the patient to prevent cancer recurrence in the patient; use of allogeneic cells collected from a matched donor in the manufacture of a vaccine for preventing cancer in a healthy subject; or use of autologous cells collected from a cancer patient in the manufacture of a vaccine to prevent cancer recurrence in the patient.

In specific embodiments the vaccine, and preferably oncoantigens present in the vaccine are treated with IFN-γ, IL-2 or both. The treatment can be carried out at any stage prior to administration of the vaccine.

In many of its aspects, the invention is practiced in a prophylactic manner in subjects who are healthy and who have normal immune system but may be at some risk of developing a specific cancer. Other embodiments of the invention can be employed to protect against a wide spectrum of cancers and are particularly useful in immunizing patients who may be at risk for developing cancer but for whom the exact type of cancer is difficult to predict. Successful immunization can be determined by straightforward determinations and the invention can be applied in clinical trials, preferably of a controlled category where data can be collected and analyzed using known statistical methods.

In other aspects, the invention is practiced in patients with a specific cancer that is in remission achieved by surgery, chemotherapy, radiation therapy and/or other means using vaccines containing tumor-associated antigens. The approach is simple and its success or failure easy to assess.

Advantages of some aspects of this invention relate to the way to make cancer vaccines more immunogenic thus improving chances for success of vaccination.

Furthermore, embodiments of the invention improve success of vaccination of patients in remission of their cancer by administration of autologous immune competent cells.

The above and other features of the invention including various novel details of construction and combinations of parts, and other advantages, will now be more particularly described and pointed out in the claims. It will be understood that the particular method and device embodying the invention are shown by way of illustration and not as a limitation of the invention. The principles and features of this invention may be employed in various and numerous embodiments without departing from the scope of the invention.

### DETAILED DESCRIPTION OF THE PREFERRED EMBODIMENTS

Cancers manifest themselves through malignant tumors of potentially unlimited growth that can expand locally by invasion and systemically by metastases. Thus cancer cells can invade nearby tissues and can spread to other parts of the body through the bloodstream and lymphatic system. Suffering or having cancer refers to an abnormal bodily state marked by such tumors. As used herein, cancer refers to diseases in which abnormal cells divide without control.

There are several main types of cancer. Carcinoma, for instance, is cancer that begins in the skin or in tissues that line or cover internal organs. Sarcomas begin in bone, cartilage, fat, muscle, blood vessels, or other connective or supportive tissue. Leukemia is cancer that starts in blood-forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In one aspect, the invention relates to a prophylactic method aimed at preventing cancer. It is particularly applicable in preventing onset and/or development of solid tumors such as can manifest in the breast, ovary or prostate. This aspect of the invention also can be practiced with respect to lung, pancreas, skin, liver, stomach, kidneys, uterus, appendix and brain. Other cancers also may be prevented.

In many aspects, the method is applicable in subjects who do not have cancer, as determined by diagnostic techniques known in the art, and who preferably are healthy. In specific examples the subjects have an intact immune system. At a minimum, an intact immune system is demonstrated by normal white blood cell count and function and normal count and function of T and B lymphocytes. Additional indicators such as amount and function of immune globulins also can be determined and preferably found to be within a normal range.

The subjects can be human or non-human, e.g., pets, or other animals.

In many embodiments, the subject(s) selected are at risk for presenting with cancer in the future.

Many risk factors for cancer development are currently known and others are being established. Among them, family history is one criterion currently used by physicians to recommend customized monitoring programs such as increased frequency of mammograms and so forth. In breast cancer, for instance, the method can be practiced in healthy patients whose mother or grandmother had (or have) breast cancer.

Other suitable subjects are those whose parents have both had (or have) cancer. Subjects who smoke or subjects who are or have been exposed, e.g., through their occupation, to asbestos or other compounds known to potentially cause cancers, for instance cancer of the lung. Chimneysweepers or factory workers handling dusts such as in the cement industry, in facilities that use fine silica or carbon particles, organic or polymeric materials, and others people routinely exposed to materials that are known or are suspected for causing cancers also can be selected.

Another category suitable for vaccination are the people with significant sun exposure due to their occupation, e.g., farmers and construction workers in sub-tropical and tropical climates, as well as patients with congenital and other nevi and other skin lesions, known to have higher incidence of malignant transformation.

Patients infected with hepatitis B, hepatitis C, and human papilloma viruses as well as patients infected with H. pylori bacteria are at higher risk of developing cancers and could be another category of subjects suitable for vaccination.

A depressed immune system, such as can be found in HIV-positive or AIDS patients, transplant recipients, geriatric subjects and so forth, can be another criterion for selecting suitable subjects.

Future research may establish other correlations such as alcohol consumption, eating, sleeping, travel patterns and others. For instance, healthy subjects who treat or live with cancer patients (possibly for long periods) may be found to be more at risk for developing cancers than others.

With developments in genomic research, correlations are being established between genetic profiles and risk for developing any number of diseases, including specific cancers. Accordingly, a powerful tool for selecting suitable subjects can be based on known or future laboratory or clinical techniques established for assessing existing genetic indicators or for monitoring changes in such indicators.

In one example, inherited alterations in the genes called BRCA1 and BRCA2 are involved in many cases of hereditary breast and ovarian cancer. Women with an altered BRCA1 or BRCA2 gene are 3 to 7 times more likely to develop breast cancer than women without alterations in those genes. Men with an altered BRCA1 or BRCA2 gene also have an increased risk of breast cancer (primarily if the alteration is in BRCA2), and possibly prostate cancer. Alterations in the BRCA2 gene have also been associated with an increased risk of lymphoma, melanoma, and cancers of the pancreas, gallbladder, bile duct, and stomach in some men and women.

The method also can be practiced in entirely healthy subjects who are not known to be at risk.

Another aspect of this invention applies to immunotherapy of patients who already have cancer, e.g., a cancer that manifests through solid tumors, such as described above. One example would be a patient who has achieved remission from his/her cancer through surgery, chemotherapy, and/or radiation, or by other means. This aspect of the invention provides for prevention of cancer recurrence in such a patient.

The subject selected is administered a formulation which consists of, consists essentially of or comprises a vaccine.

The vaccine can be obtained from one or more sources such as whole cancer cells and lysed cells, gene-modified tumor cells, heat-shock proteins, peptides, naked DNA, ex vivo dendritic cells, and others.

It is known that some types of cancers share common tumor antigens. Breast and ovarian cancers, for example, share tumor antigens such as HER-2/neu. Nevertheless, each specific type of cancer also can have type-specific antigens.

One of the best candidates for the vaccine is a prototypic cancer antigen NY-ESO-1 that is expressed naturally in a wide variety of cancer types but not in healthy tissues except for immune-privileged cells found in the testes. The immune system responds spontaneously to the presence of this antigen, as evidenced by the presence of NY-ESO-1-specific antibodies and T cells in patients with cancers expressing NY-ESO-1, and an integrated immune response to this antigen can be artificially induced through vaccination. Vaccines can be composed of a single NY-ESO-1 peptide, overlapping peptides, and whole NY-ESO-1 protein. The NY-ESO-1 antigen, which is owned by the Ludwig Institute for Cancer Research, is produced at a GMP facility in Ithaca, N.Y. and at other locations globally.

In one embodiment of the invention, a subject at risk for a particular cancer is immunized with a vaccine obtained from one or more “matched” donor(s), i.e., donor(s) having the same cancer. For instance, a matched donor for a subject at risk for developing prostate cancer is a patient suffering from prostate cancer. In another case, a woman with a strong family (mother and grandmother) history of breast cancer is vaccinated with a vaccine obtained from one or more matched donors, i.e., patients having breast cancer.

Another embodiment relates to subjects at risk for a wider spectrum of cancers, e.g., subjects with a depressed immune system. For subjects who are at risk for developing cancer but for whom it is difficult or impossible to predict the exact type of cancer, the vaccine can be obtained from donors with different cancers, e.g., melanoma, breast, lung, leukemia, lymphoma, myeloma and so forth.

In some implementations, a vaccine is prepared using a combination or “cocktail” of cells or cell extracts. These cells or cell extracts are obtained from two or more groups of patients, each group including one or more patient(s) with the same type of cancer, patients in different groups having different cancers. For instance a vaccine could be prepared from cells obtained from the following groups of donors: Group A: cancer of the breast; Group B: glandular cancer; and Group C: skin cancer. Similar cocktail vaccines can be obtained from other combinations of cancer donors.

Cocktail type vaccines are particularly useful in healthy subjects, immuno-depressed subjects or in other cases when a subject may present with any type of cancer.

The cells used as the vaccine or as precursors to the vaccine preferably are live cells and can be used in cultures, as known in the art. Frozen cells that are preserved under cryogenic conditions also can be used. In one example, the vaccine is prepared from cells frozen in liquid nitrogen and restored at a later time.

To reduce or minimize variations from one batch of vaccine to the next, cells can be collected from two or more patients suffering with the same type of cancer, e.g., prostate cancer.

In specific embodiments, the vaccine is an extract obtained from the membrane of the allogeneic cells. In other embodiments, the vaccine is an extract obtained from the cytoplasm of the allogeneic cancer cells. Either or both extracts can be administered, as further described below.

The vaccines can also be prepared from autologous tumors removed during surgery and later administered to the same patient.

A suitable technique for preparing cells for a cancer vaccine is described, for example, in the U.S. Pat. No. 5,635,188, issued to Bystryn on Jun. 3, 1997, the teachings of which are incorporated herein by reference in their entirety. Tumor cells are incubated at a concentration of 2×10⁶/ml serum-free RPMI 1640 medium. After 3 hours at 37° C. the medium was collected and the cells were removed by centrifugation at 2,000 G for 10 minutes, and larger particles were removed by centrifugation at 12,000 G for 15 minutes. Medium was then concentrated 10-fold by vacuum ultrafiltration and made up to a final concentration. In some cases vaccines were prepared with further treatment including the addition of a surfactant and 0.02% sodium azide, and then ultracentrifuged at 100,000 G for 90 minutes. The supernatant was dialyzed at 4° C. against normal saline with 0.02% sodium azide and made up to the desired protein concentration by the addition of normal saline, passed through a 0.1 μm Millex Millipore filter to remove microorganisms; and 0.5 ml aliquots dispensed into sterile, pyrogen-free glass vials. The vials were stored at ±70° C. until used.

The vaccine includes one or more cancer (onco) antigens, e.g., whole cells (both autologous and allogeneic) or cell extracts (lysates, membranes and heat-shock proteins), gene-modified cancer cells, cancer cells fused to antigen-presenting cells, proteins, synthetic polypeptides, purified cancer antigens (natural or recombinant), RNA, “naked” DNA, enzymes, cellular mass and so forth.

The antigens can be combined with immunocompetent tissue such as cytotoxic T lymphocytes, natural killer cells, macrophages, bone marrow or components thereof and so forth.

Incubation, inactivation and other suitable techniques can be employed to prepare the vaccine.

In some cases the vaccine is prepared using specific compounds identified and isolated from the allogeneic cancer cells. In other cases, such identification and/or isolation steps are not carried out and subjects receive cells or cell extracts as obtained from the donors.

In specific examples, the active agent present in the vaccine is NY-ESO-1. The NY-ESO-1 gene encodes a member of the cancer/testis (CT) family of human tumor-associated antigens (TAA). As described, for instance, by E Schultz-Thater in the article, “NY-ESO-1 Tumour Associated Antigen is a Cytoplasmic Protein Detectable by Specific Monoclonal Antibodies in Cell Lines and Clinical Specimens,” published in the British Journal of Cancer (2000), 83: 204-208, the gene product was identified in lysates of tumor cell lines as a 22 kDa protein using specific monoclonal antibodies (mAb) and NY-ESO-1 specific mAbs have been used to recognize the target molecule in cytospin preparations and in sections from clinical tumor specimens. NY-ESO-1 specific mAbs have been used to identify NY-ESO-1 TAA in melanoma cell lines expressing the specific gene as a cytoplasmic protein, sharing the intracellular location of most MAGE TAA. Other well established antigens that can be used for vaccination include MAGE-3, NY-BR-1, SSX-2, NY-CO-58, MELAN-A, and others.

To increase its immunogenicity, the vaccine can be treated with gamma interferon (IFN-γ) and/or, interleukin-2 (IL-2). For example, oncoantigens are mixed with Hank's solution and centrifuged at 800 G for 10 minutes to form a precipitate. Erythrocytes are hemolysed, e.g., in the presence of a solution of 0.83% hydrochloric acid. Precipitate is then washed twice in Hank's solution and incubated in human IFN-γ and/or IL-2 in the amount of 1 milliliter of the cytokine per 5×107 cells at 4° C. for 18 hours. Supernatant is then removed and the precipitate is combined with culture medium No. 199-0.2 ml per 5×107 cells.

IFN-γ is an important mediator of adaptive and innate immunity and plays critical role in promoting both protective immune responses and immunopathological processes. IL-2 has no direct impact on the tumor cells but mediates antitumor activity through the modulation of the host's immune response.

The vaccine can be formulated to include other compounds that can prevent development of cancer in a healthy individual. In one example, the vaccine includes tumor suppressor genes, which can already be present in the allogeneic cells described above or can be introduced separately.

The vaccine can be present in a formulation. In addition to the vaccine, the formulation can include a suitable medium, selected, for example, to provide ease of administration, increased bioavailablity, sustained release properties, and so forth.

The formulation can include adjuvants, vectors, chemotherapeutic drugs, other vaccines, immune modulators, cytokines, chemokines, and/or or other suitable ingredients.

The vaccine is present in the formulation in a suitable amount, e.g., in an amount within the range of from about 1 percent by weight (wt %) to about 100 wt %.

Suitable administration routes include injection, e.g., intradermal, subcutaneous, intramuscular, intraperitoneal, intravenous, transdermal, oral, rectal, vaginal, ocular as well as others known in the medical and pharmaceutical arts.

The amount of cell extract for a single administration by intradermal injection can be in the range of from about 0.1 to about 0.2 ml. Larger volumes can be administered subcutaneously or intramuscularly.

The vaccine preferably is administered more than once, for instance it can be administered on a monthly basis. Other schedules, e.g., once weekly or once every two months, can be employed as well. Healthy subjects can receive the vaccine at frequencies less than once a month and the vaccine can be administered for a period of, e.g., five years or longer. Patient diagnosed with cancer preferably begin vaccine administration after remission is observed, typically several months, e.g., ten to twelve months, into remission.

In patients who have been diagnosed with cancer and are immunized against cancer recurrence, the vaccine can be administered in combination with bone marrow, e.g., the patient's own, and/or T lymphocytes, preferably the patient's own. Bone marrow can be obtained by standard aspiration practices and known techniques can be employed to collect lymphocytes.

In further implementations of the invention, prior to administration, the bone marrow and/or T lymphocytes are treated with IFN-γ and/or IL-2. Methods of obtaining bone marrow and T lymphocytes are well known in the art. A method of incubation of peripheral blood mononuclear cells with IL-2 has been described in U.S. Pat. No. 4,690,915 issued on Sep. 1, 1987 to Rosenberg et al., the teachings of which are incorporated herein by reference in their entirety. Similar technique can be used for incubation of bone marrow and T lymphocytes in IL-2 and IFN-γ.

Preferably, in healthy patients, the vaccine is administered without administration of bone marrow and/or T lymphocytes.

In preferred aspects, the subject is monitored with respect to his or her response to the vaccine. For instance, the subject is evaluated, preferably before, during the period (e.g., weeks, months or years) spanned by the administration schedule, and/or after vaccine administration is ceased. In specific embodiments, the patient is evaluated once or at repeated intervals after each vaccine administration.

Evaluation techniques include techniques designed to determine onset of cancer and include methods for determining immunological factors in the organism. Suitable approaches are described, for example, in the article “Enumerating Antigen-Specific T-cell Responses in Peripheral Blood”, by Amy C. Hobeika, et al, Journal of Immunotherapy 28(1):63-72, 2005. This article evaluates several standard assays for detection of circulating antigen-specific T-cells in response to immunization. Another method of determining tumor status is described in the U.S. Pat. No. 6,251,603 B1 issued to Jager et al. on Jun. 26, 2001, the teachings of which are incorporated herein by reference in their entirety, and involves measuring antibodies to NY-ESO-1 tumor antigen.

Criteria for establishing a positive immunological response to the vaccine have been described in the article “Immunologic Monitoring of Cancer Vaccine Therapy: Results of a Workshop Sponsored by the Society for Biological Therapy” by Ulrich Keilholz, et al, Journal of Immunotherapy 25(2):97-138, 2002. Positive immunological response measurements will depend upon the cancer vaccine used (e.g., in a specific protein vaccine trial, antibody response, CD4 and CD8 T-cell responses are measured; in case of vaccination with modified tumor cells or with tumor-cell lysates, a limited number of vaccine antigens will be characterized and immune response to these specific antigens will be monitored.

If immunological response is not observed, is slight or is mixed in nature, e.g., if only some but not other immunological tests indicate a positive response, vaccination can be continued using the same administration regimen until a positive response is obtained. In many cases, however, dosage, type of formulation, administration frequency, delivery route and/or other parameters can be modified to elicit a positive response.

Vaccination can be terminated altogether if positive immunological response(s) cannot be elicited. Another event that can trigger termination of vaccine administration is the onset of cancer. Administration of the vaccine also can be terminated or can be suspended or interrupted for extended periods, e.g., several months, a year or longer, based on positive immunological response(s) described above. Preferably, such positive response(s) are sustained for a period of at least 5 years before the vaccination schedule is terminated or suspended.

Subjects can be monitored after vaccine administration is terminated and this evaluation can span long periods, e.g., years and can extend throughout the life of the individual. To generate statistically meaningful data, entire groups of subjects who have received the vaccine are monitored for onset of cancer or other indicators, e.g., immunological response(s) such as those described above. Monitoring can be conducted over several years, decades and preferably for the remaining life span of each subject. Control groups in which subjects matched to the group receiving the vaccine are given a placebo or nothing at all, or historical controls preferably also are monitored. Designing and carrying out controlled, e.g., single, double or triple blind, clinical trials or controlled experiments in laboratory animals are known in the art, as are approaches to randomization of a subject pool and mathematical and statistical approaches for collecting and analyzing data.

For example, patients receiving the vaccine are matched with patients receiving nothing or receiving a placebo with respect to age, gender, ethnicity, occupational exposures, genetic factors, smoking habits, and so forth.

Statistical significance between groups receiving the vaccine and control groups (e.g., placebo or groups of patients receiving neither the vaccine nor a placebo) can be determined by a technique such as the Student's t test, and many others.

For statistical evaluations, results observed in subjects receiving the vaccine also can be compared with existing data obtained or known regarding the population at large or population subgroups.

Suitable periods for observing subjects being immunized in comparison to controls for cancer onset can range from five to ten years, preferably to twenty and more, preferably to the remaining life span of the subjects and controls.

Successful immunization is demonstrated by a statistically significant difference in cancer development between subjects receiving the vaccine and controls. For example, successful immunization in subjects receiving vaccine against breast cancer can be demonstrated by a statistically lower incidence of breast cancer observed in the immunized subjects with respect to control patients over a period of time of at least five years.

In a recent consensus statement of the Cancer Vaccine Clinical Trial Working Group (CVCTWG) published in the Journal of Immunotherapy 30(1):1-15, 2007, entitled “A Clinical Development Paradigm for Cancer Vaccines and Related Biologics”, by Axel Hoos, et al, “biologic activity of a vaccine is defined as any effect of the vaccine on the target disease or host immune system using biological markers as study end points, for example, clinical, molecular, or immune response”.

Immunization techniques described herein can be accompanied by administration of immune system boosters, changes in exposure to harmful compounds, e.g., reducing or ending smoking or alcohol consumption, increased exercise, weight loss and other approaches aimed at improving health and immune responses.

Whereas in healthy subjects many biological processes include a repressor step, in cancer one or more of these repressor steps can become absent. As known in the art, for example, cancer cells escape the restraints on normal cell growth and tumors or other manifestations of the disease are observed. The onset of cancer can be accompanied by a weakening or depression of the immune system.

Normally, healthy immune system reacts to every “foreign” substance, such as bacteria, viruses, and others. In cancer, the immune system is suppressed and it does not respond appropriately to cancer cells which themselves could be immunosuppressive. It also leads to development of metastases later in the course of the disease. Sentinel lymph node metastases also lead to immune suppression within the nodes themselves.

In yet another aspect of the invention, a patient with solid tumors receives a vaccine prepared using the patient's own (or autologous) tumor cells. They can be obtained during surgical resection of the patient's tumor. Single cell suspensions are produced using enzymatic digestion and then cultured in tissue culture serum-free medium.

Cells can be preserved using known techniques, for example, under cryogenic conditions, e.g., below −196 degrees centigrade (° C.), for instance at liquid nitrogen temperatures.

Many immunological responses against cancer rely on T-cytotoxic lymphocytes. Cancer cells, however, can be covered with humoral antibodies, e.g., IgG, impeding or preventing lymphocyte attack to destroy cancer cells.

Yet another aspect of the invention relates to removing antibodies that cover cancerous cells, thereby facilitating or restoring lymphocyte activity against cancer cells. Techniques that can be employed to remove antibodies that cover cancer cells include but are not limited to the use of anti-IgG antibodies. For example, IgG antibodies can be removed by injection, e.g., using anti-IgG antibodies, locally in the cancer, for instance breast cancer.

Treatment of oncoantigens with IFN-γ and/or IL-2 can increase T-cell response to cancer cells. In addition, treatment of a patient's lymphocytes and bone marrow with IF-γ and/or IL-2 can increase cytotoxic activity of the cells against cancer cells. Cytokines such as IFN-γ and/or IL-2 are well known in the art. A method of incubation of peripheral blood mononuclear cells with IL-2 has been described in U.S. Pat. No. 4,690,915 issued on Sep. 1, 1987 to Rosenberg et al., the teachings of which are incorporated herein by reference in their entirety. Without wishing to be held to a particular interpretation regarding any aspect of the invention, it is believed that if or when cancer develops, cancer cells will encounter antibodies or immune lymphocytes capable of destroying the cancer cells.

### EXAMPLES

### Example 1

### Immunization with Live Native Allogeneic Leukemic Cells During Acute Periods (S. Skurkovich et al. Nature. 1969; 223:509-511)

Immunization was performed in the operating room. Active immunization was performed using live allogeneic leukemic cells from peripheral blood intravenously as well as bone marrow cells intramuscularly. Cells from one patient were injected into another and the cells from the second patient were administered to the first one. We treated 12 children (6 pairs) age three to ten years with different morphological varieties of acute leukemia. The percentage of leukemic cells in peripheral blood ranged from 40-80%. Course of active immunizations has lasted from six to 28 days and included two to five intravenous administrations of leukemic cells (from 5×10⁶ cells to 4.1×10⁹ for each administration) every two to four days. Passive immunization was performed eight to 15 days after active immunization with plasma and leukocytes obtained from patients by plasmapheresis. Each child was passively immunized two to three times in intervals of one to four days. Of the 12 patients who underwent these active immunizations, eight experienced a positive effect. We have observed a decrease in the number of leukemic cells in peripheral blood from day four to day seven. We have observed two to three times decrease in the number of leukemic cells in the peripheral blood and by day 14 to 28, they have reached 0-16%. Thus, three out of twelve patients who underwent active immunization with leukemic cells went into complete remission and five to partial improvement in hematological indices. After performance of immunizations, all children except for one, received chemotherapy as well. One patient did not undergo chemotherapy due to parental refusal. In the period of complete remission, active immunizations with leukemic cells were continued (total number of 30 intramuscular injections of leukemic cells twice a week) and it was stopped five months after the beginning of treatment. Later, all of these children died at different times despite receiving chemotherapy. These results have shown that there are immunocompetent cells in the organisms of patients with acute leukemia in the acute phase that can react to leukemic antigens but that this immunocompetent system is so weak that it cannot overcome these leukemic antigens. These results were broadened in 54 patients both children and adults with acute leukemia immunized with live leukemic cells simultaneously with chemotherapy. These immunizations in the acute period of acute leukemia lead only to a certain temporary success because these patients did not have mature immunocompetent system.

### Example 2

### Immunization of Children in Remission of Acute Leukemia with Allogeneic Live Leukemic Cells (S. Skurkovich et al., to be presented at the World Cancer Congress, Jun. 12-17, 2008, Shanghai, China)

We treated 54 children with ALL who have achieved complete remission after standard-for-that-time chemotherapy: 27 received standard maintenance chemotherapy and immunotherapy and 27 received chemotherapy alone. Immunotherapy consisted of a long-term administration of viable cryopreserved at −196° C. and thawed leukemic cells without immunoglobulins on their surface. All patients younger than 7 years of age who received immunotherapy starting in the first several months of their remission did not exhibit any therapeutic effect. Immunotherapy was effective in children younger than 7 if it was initiated after 1-1, 5 years of remission. In children over 7 years of age, if immunotherapy was started after 6 months of remission, it led to its stabilization in all patients. After 5 years of remission all therapy was stopped and they are living normal lives; some have already had their own healthy children and one has a grandchild. As of the end of 2007, 8 out of 19 (42.1%) immunized children had achieved remission lasting over 10 years (mean 32.5 years) and are considered cured. Sera of these patients contained cytotoxic antibodies to leukemic cells and donor lymphocytes. Intrathecal administration of 1-2 ml of these sera to patients with neuro-leukemia resistant to chemotherapy led to sharp decrease in pleocytosis. These results demonstrate that condition of the immune system is critical for the success of immunization against cancer. Comprehensive immunologic workup of patients who received immunotherapy in the period of remission showed signs of stimulation of humoral and cell-mediated immunity in comparison with patients who were on chemotherapy alone. In the blood of patients immunized with live leukemic cells, we have observed gradual (over one or two years) increase in humoral and cell-mediated immunological indices directed towards antigens of administered leukemic cells; cytotoxic (in C-dependent cytotoxic reaction) and non-cytotoxic (by method of indirect immunofluorescence) antibodies as well as cytotoxicity of lymphocytes. In the majority of cases, we have found cross-reactivity with antigens of leukemic cells that have not been used for immunotherapy as well as donor lymphocytes that have been found in the blood of patients one to three years after the beginning of immunotherapy. These results proved that the antibodies that were stimulated in patients receiving immunotherapy with live allogeneic leukemic cells were active but also showed a new direction in the immunotherapy or neuroleukemia which is resistant to chemotherapeutic preparations. Retrospectively, we have also observed positive effects of the development of cytotoxic antibodies in the blood of immunized patients. Almost all patients without recurrence of leukemia had increasing titers of cytotoxic antibodies to leukemic cells (in smaller titer towards allogeneic lymphocytes). All children who relapsed had absent antileukemic cytotoxic antibodies in their sera. Thus, our investigation demonstrates that when immune system is completely restored and becomes intact the immunization leads to complete recovery of the patient. At the same time all patients who received chemotherapy alone died.

### Example 3

A rational approach to immunotherapy of solid tumors is described below. In this approach immune cells such as lymphocytes, bone marrow and possibly lymph nodes are obtained periodically and cryopreserve at −196° C. Cell collection is conducted during the stage of remission after surgery, chemotherapy, and/or radiation therapy and these tissues can be used together with oncoantigens in order to prevent recurrence of patient's cancer. First of all, since there are frequently blocking antibodies found in solid tumors and these antibodies prevent cytotoxic action of immune lymphocytes, for some patients with solid tumors it is important to administer antibodies, preferably monoclonal humanized or human, to IgG.

For broad use in vaccination against solid tumors, it is useful to create a bank of live tumor cells preserved at −196° C. Tumors that are removed in auto- and allo-systems during surgeries can be sterilized, trypsinized and cryopreserved at −196° C.

Immunocompetent cells obtained in the remission period should be given after sustained remission has been achieved, and these cells should be given multiple times together with standard prototypic tumor antigens or antigens obtained from the same patient. It is possible to use not only autologous tumor cells but pooled allogeneic tumor cells as well. Immunocompetent and tumor cells can be administered after preliminary contact with each other and immediately. It is possible to create a new immunological system in patients with tumors and leukemia. In an approach to the treatment of tumors and leukemia, as many as possible leukemic and tumor cells are disintegrated or eliminated.

If, after treatment with surgeries, radiation and/or chemotherapy, immunotherapy or others means, even a small number of tumor cells remain, thus indicating that immunocompetent system of patients cannot eliminate them, then relapse of the disease is inevitable.

### Example 4

There are groups of people who have genetic predisposition to cancers making them more likely to develop tumors. Genetic markers of different cancers are increased in certain ethnic groups, for example, Ashkenazi Jewish women are more genetically predisposed to breast cancers compared to other women. We think that these individuals could be a prime target for immunizations with tumor antigens while they are healthy. They would need to be immunized for long periods of time with tumor antigens under immunological supervision. It is very possible that this approach would prevent the development of cancers in these individuals. However, in order to undergo this immunization they have to have completely healthy immune system. Multiple tumor antigens that have preserved their antigenic activity and that are safe for the patients can be used.

### Example 5

Patients in remission from their solid cancer, achieved through surgery and/or chemotherapy and radiation are selected for vaccination. Only patients whose remission has lasted for at least 10-12 months without any signs of recurrence are eligible. Patients undergo bone marrow aspiration using standard technique and their bone marrow is stored. These procedures are repeated every 4-6 months. Some patients undergo leukapheresis to collect their peripheral blood lymphocytes either instead or in addition to the collection of their bone marrow. A standard oncoantigen, e.g., NY-ESO-1, treated with IFN-γ and/or IL-2 (as described above), is administered subcutaneously at a dose of 400 μg of protein, every 3 to 4 weeks. Bone marrow and/or T lymphocytes are incubated with IFN-γ and/or IL-2 (as described above) and administered intravenously at the same time as oncoantigens. The first bone marrow infusion is administered about 2 months after its collection. Thus, each patient will be receiving monthly immunizations with an oncoantigen and/or an infusion of their own bone marrow or their own peripheral blood immune lymphocytes. Cells collected with each bone marrow aspiration will be used for 4-6 months until a new aspiration is performed. This schedule is continued until at least 5 years of remission have been achieved. This will create a permanent anti-tumor immunity. Occasionally, matched bone marrow and/or T lymphocytes can be used as well.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for cancer prevention, the method comprising:
a. selecting a healthy subject who is cancer-free, has never been diagnosed with cancer and has an intact immune system; and
b. administering to the subject a vaccine, wherein an oncoantigen present in the vaccine is pre-treated with IFN-γ, IL-2 or a combination thereof.

2. The method of claim 1, wherein the patient is at risk for developing cancer in the future.

3. The method of claim 1, further comprising monitoring the subject for onset of cancer indicators.

4. The method of claim 1, wherein the oncoantigen is NY-ESO-1.

5. The method of claim 1, wherein the vaccine includes whole allogeneic cells, extracts from allogeneic cell membranes, extracts from allogeneic cell cytoplasm, polypeptides or DNA.

6. A method for preventing cancer recurrence, the method comprising:
a. selecting a cancer patient who has remained in remission from a solid cancer for at least ten months;
b. collecting bone marrow, T lymphocytes or both from the cancer patient; and
c. administering to the patient, at intervals of at least three weeks:
(i) a vaccine that includes an oncoantigen; and
(ii) a portion of the bone marrow, of the T-lymphocytes or of a combination thereof,
thereby preventing cancer recurrence.

7. The method of claim 6, wherein the oncoantigen is NY-ESO-1.

8. The method of claim 6, wherein one or more of the oncoantigen, the bone marrow or the T lymphocytes is pre-treated with IFN-γ, IL-2 or both.

9. The method of claim 6, further comprising storing the bone marrow, T lymphocytes or both for at least two months.

10. The method of claim 6, wherein step (b) is repeated and administration of (i) and (ii) continues until the patient achieves at least five years of remission.

11. The method of claim 6, wherein the vaccine is administered subcutaneously and the bone marrow, T lymphocytes or both are administered intravenously.

12. The method of claim 6, wherein the vaccine includes whole tumor cells or extracts thereof, polypeptides, DNA or any combination thereof.

13. A method for preventing cancer recurrence, the method comprising:
a. selecting a patient with solid cancer who is in remission for at least ten months; and
b. administering to the patient a vaccine that includes, or is obtained from, autologous cancer cells, in combination with bone marrow, T lymphocytes, or both, thereby preventing cancer recurrence, wherein the bone marrow, T-lymphocytes or both are collected from the patient.

14. The method of claim 13, wherein one or more of the vaccine, bone marrow and T lymphocytes are pretreated with IFN-γ, IL-2 or a combination of IFN-γ and IL-2.

15. A method for conducting an experiment in animals or in a clinical trial, the method comprising:
a. selecting a group of healthy subjects who have intact immune system and who are at risk for developing a specific type of cancer in the future;
b. administering to the healthy subjects a vaccine containing NY-ESO-1 pre-treated with IFN-γ, IL-2 or both, to obtain an immunized group;
c. monitoring the immunized group with respect to immune indicators or with respect to onset of said specific type of cancer; and
d. comparing the immunized group with a group that has not received the vaccine with respect to incidence of said immune indicators or the incidence of the onset of said specific type of cancer.
